(19) United States
(12) Patent Application Publication
(10) Pub. No.: US 2026/0115464 A1
(43) Pub. Date: Apr. 30, 2026

(54) APPROACHES TO DETECTING REMOVAL OF NEUROMODULATION DEVICES AND MITIGATING EFFECTS OF THE SAME

(71) Applicant: Hinge Health, Inc., San Francisco, CA (US)

(72) Inventors: Samuel Corbly House, Portland, OR (US); Michael Shyh-Yen Ho, San Francisco, CA (US)

(21) Appl. No.: 19/372,156

(22) Filed: Oct. 28, 2025

Related U.S. Application Data
(60) Provisional application No. 63/713,498, filed on Oct. 29, 2024.

(51) Int. Cl. A61N 1/36; A61N 1/04

(52) U.S. Cl. CPC A61N 1/36021; A61N 1/0456; A61N 1/0492; A61N 1/36031

(57) ABSTRACT

A neuromodulation device including a first electrode via which an electrical signal is applied to the skin of a living body. The device further includes a second electrode at which the electrical signal is received and a gel pad that is electrically connected with the first and second electrodes. The device further includes a controller configured to generate the electrical signal that is applied to the skin of the living body at the first location via the first electrode, measure the electrical signal received at the second electrode and produce an analog output that is representative of the measured electrical signal, establish a change in resistance between the first and second electrodes, determine whether the gel pad is being removed from the skin of the living body based on the change in resistance, and halt generation of the electrical signal.

### CROSS-REFERENCE TO RELATED APPLICATION

The application claims priority to U.S. Provisional Pat. App. No. 63/713,498 filed Oct. 29, 2024, titled Approached to Detecting Removal of Neuromodulation Devices and Mitigating Effects of the Same, and is hereby incorporated by reference in its entirety.

### TECHNICAL FIELD

Various embodiments relate to electrical neuromodulation. More particularly, methods and devices herein provide electrical neuromodulation for treating symptoms of chronic and acute pain as well as other conditions.

### BACKGROUND

Pain is the mental manifestation of a neurological response to various physiological and psychological ailments. Pain serves as a warning of physical injury or biological dysfunction. Sometimes pain persists much longer than it takes for the healing of the initial injury to occur, and may be very difficult to alleviate. The most common pain relief methods employ drugs (e.g., opioids) that act to block neurotransmission pathways within the body. Often, such drugs are not effective for pain relief over the long term or produce unacceptable side effects. Consequently, various forms of electrical stimulation, such as spinal cord stimulation (SCS) and transcutaneous electrical nerve stimulation (TENS), have also been employed to alleviate pain.

SCS is effective but is an invasive procedure and has all the typical risks associated with implantable devices, as well as the risk of serious damage to the spinal cord. Conventional neuromodulation devices are not effective in all patients due to the difficulty in picking effective settings, and they may produce effects that only last during stimulation and do not produce long-term pain relief. Moreover, many patients find conventional TENS at therapeutically effective levels to be uncomfortable.

### BRIEF DESCRIPTION OF THE DRAWINGS

FIG. 1 illustrates a conventional example of a neuromodulation device for applying TENS.

FIG. 2 is a block diagram illustrating modules of a neuromodulation device, according to some embodiments.

FIG. 3 is a block diagram illustrating phases of an electrical pulse output from a neuromodulation device, according to some embodiments.

FIG. 4 is a pulse waveform diagram illustrating phases of one variation of an electrical pulse output by a neuromodulation device as a simplified waveform, according to some embodiments.

FIG. 5 is a block diagram illustrating an overview of the circuitry of a neuromodulation device according to some embodiments.

FIG. 6A is a circuit diagram illustrating the current measurement portion of the feedback circuitry of the neuromodulation device, according to some embodiments.

FIG. 6B is a circuit diagram illustrating a unity gain difference amplifier portion of the feedback circuitry, according to some embodiments.

FIG. 6C is a circuit diagram illustrating a voltage measurement portion of the feedback circuitry, according to some embodiments.

FIG. 7 is a graph illustrating sample resistance measurements, according to some embodiments.

FIG. 8 is a flow diagram for a method of detecting the removal of a neuromodulation device, according to some embodiments.

Various features of the technology described herein will become more apparent to those skilled in the art from a study of the Detailed Description in conjunction with the drawings. Various embodiments are depicted in the drawings for the purpose of illustration. However, those skilled in the art will recognize that alternative embodiments may be employed without departing from the principles of the technology. Accordingly, although specific embodiments are shown in the drawings, the technology is amenable to various modifications.

### DETAILED DESCRIPTION

Many conventional neuromodulation devices offer limited effectiveness in patients for various reasons. One reason is that the physical construction of many neuromodulation devices means that the devices are not effective in offering a user of the device pain relief while also remaining comfortable and not interfering with daily activities. For example, many conventional neuromodulation devices are bulky or difficult to wear, which leads to many users experiencing pad peel events. A pad peel event is felt as an electric shock and occurs when the pad containing the neuromodulation device's electrode is removed while the neuromodulation device is actively treating the user by emitting an electrical signal into the user's body. The electrode supplies the electrical signal, such as a current or voltage, to the pad. The pad is placed on the user's skin and distributes the electrical signal to the user's body.

Neuromodulation devices typically need to supply high amounts of current to reach the level proven to be therapeutically effective. The current density is amplified during a pad peel event because the contact area of the pad is reduced while the neuromodulation device is on and emitting a current into the user's body. The reduced contact area causes the current density to rise, leading to the painful zaps or shocks felt by the user as the electrode is removed. These pad peel events often occur accidentally while wearing the neuromodulation device. For example, while the neuromodulation device is active, a pad peel event can occur when a user removes or adjusts the pad or when a pad slowly peels from a user's skin due to a loss of adhesion in the pad movement by the user. Conventional neuromodulation devices do not utilize methods of detecting these pad peel events to prevent users from experiencing painful shocks caused by a pad peel event.

Introduced here are neuromodulation devices and methods that overcome the deficiencies of conventional neuromodulation devices to provide effective pain relief while preventing the discomfort caused by pad peel events experienced while using conventional neuromodulation devices. Pad peel events can be detected using sensors such as an accelerometer placed on the pad, but the user's movement can generate signals that indicate the existence of a pad peel event when one does not exist.

As further discussed below, the neuromodulation devices introduced here overcome the issues of using a motion sensor to detect a pad peel event, along with deficiencies of conventional neuromodulation devices, by using a processor and analog circuitry to determine the existence of a pad peel event. Because the shrinking contact area of the electrode that causes the painful shock also results in a proportionally increased resistance between the electrode and the user, the neuromodulation device can detect the existence of a pad peal event by measuring a change in resistance. The neuromodulation device's analog circuitry includes feedback hardware used to return a portion of the electrical signal output back through the circuit as an input. This enables the neuromodulation device to measure the current sent through a user's skin between the two electrodes. The measured current value is transmitted to the processor. In some embodiments, the current value is converted from an analog output to a digital output using an analog-to-digital converter. Using the processor and Ohm's law, the neuromodulation device calculates a resistance value based on the measured current value and a known voltage. An average resistance value is used because the base resistance level for a regular pad connection varies depending on the type of gel pad used and the user's natural resistance. Using the resistance delta enables the detection of pad peel events without needing to calibrate the neuromodulation device to each user and pad type. The average resistance value is used as the baseline resistance level for normal pad connection conditions. When a baseline resistance level is determined, the neuromodulation device monitors the current level received from the feedback hardware to determine a spike in resistance and, therefore, a pad peel event. The spike in resistance is compared against the average resistance during normal pad connection conditions to determine the existence of a pad peel event. Conventional neuromodulation devices do not contain feedback hardware, meaning conventional neuromodulation devices cannot use a measured resistance to detect a pad peel event or even whether a pad is placed on a user. Given the neuromodulation device can measure a resistance delta, a neuromodulation device according to embodiments herein can:
Enable the detection of pad peal events for different users using different gel pads;
Prevent painful shocks to users by stopping the current flowing to the electrodes when a pad peel event is detected; and
Notify a user when a pad peal event is occurring.

Embodiments herein may be described in the context of a “user”. A “user” may be a living body receiving electrical signals from an electrode or transmitting signals to an electrode. In some embodiments, a user may be human. In other embodiments, a user may be an animal. Accordingly, electrodes and neuromodulation devices according to embodiments herein may be used in connection with any living body.

In some embodiments herein, a neuromodulation device may be a self-contained device. For example, in some embodiments, a neuromodulation device may include a housing that houses a first electrode and a second electrode. In some embodiments, a first electrode and second electrode may have a fixed relationship to one another. In other embodiments, a first electrode and a second electrode may be independent from one another, such that the first electrode and the second electrode are able to be independently positioned on a user's skin. In some embodiment embodiments, a first electrode and a second electrode may be connected to a controller via respective flexible wires.

Some embodiments herein are discussed with reference to a flow of current from a first electrode to a second electrode. In other embodiments, current may flow from a second electrode to a first electrode. As a part of TENS treatment, a direction of current flow may be reversed during a treatment session. Accordingly, electrodes described herein may be employed interchangeably as a source electrode or a sink electrode.

Embodiments may be described in the context of computer-executable instructions for the purpose of illustration. However, aspects of the approach could be implemented via hardware or firmware instead of, or in addition to, software.

### Terminology

References in the present disclosure to “an embodiment” or “some embodiments” mean that the feature, function, structure, or characteristic being described is included in at least one embodiment. Occurrences of such phrases do not necessarily refer to the same embodiment, nor are they necessarily referring to alternative embodiments that are mutually exclusive of one another.

Unless the context clearly requires otherwise, the terms “comprise,” “comprising,” and “comprised of” are to be construed in an inclusive sense rather than an exclusive or exhaustive sense. That is, in the sense of “including but not limited to.” The term “based on” is also to be construed in an inclusive sense. Thus, the term “based on” is intended to mean “based at least in part on.”

The terms “connected,” “coupled,” and variants thereof are intended to include any connection or coupling between two or more elements, either direct or indirect. The connection or coupling can be physical, logical, or a combination thereof. For example, elements may be electrically or communicatively coupled to one another despite not sharing a physical connection.

The term “module” may refer broadly to software, firmware, hardware, or combinations thereof. Modules are typically functional components that generate one or more outputs based on one or more inputs. A computer program may include or utilize one or more modules. For example, a computer program may utilize multiple modules that are responsible for completing different tasks, or a computer program may utilize a single module that is responsible for completing all tasks.

When used in reference to a list of multiple items, the word “or” is intended to cover all of the following interpretations: any of the items in the list, all of the items in the list, and any combination of items in the list.

### Overview of Conventional Neuromodulation Device

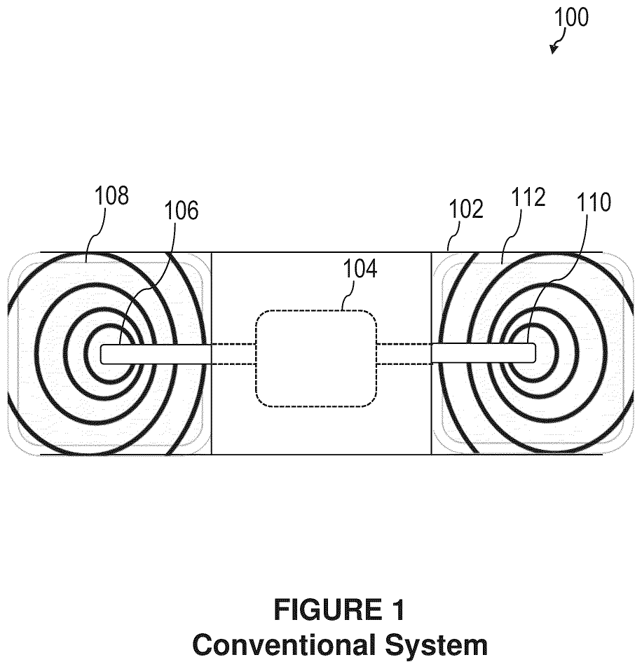

FIG. 1 illustrates a conventional example of a neuromodulation device 100. The neuromodulation device 100 is configured to apply an electrical signal to a user of the neuromodulation device to inhibit transmission of pain signals through the nervous system to the brain. The neuromodulation device 100 is configured as a patch 102 configured to be worn on the skin of a user. The patch 102 may include an adhesive material allowing the neuromodulation device 100 to adhere to the skin. The neuromodulation device 100 includes a controller 104 configured to control generation of the electrical signal. The controller 104 may be powered by an onboard power source, such as a battery.

As shown in FIG. 1, the neuromodulation device 100 includes a source electrode 106 disposed in a first gel pad 108. Electrical current is configured to flow through the source electrode 106, through the gel pad 108, and into the skin of the user of the neuromodulation device 100. The neuromodulation device 100 also includes a sink electrode 110 disposed in a second gel pad 112. Current is configured to flow from the source electrode 106, into a user's skin, and to the sink electrode 110. The passage of this electrical current is configured to disrupt pain signals in the neurological system of the user, thereby alleviating pain. The first gel pad 108 and the second gel pad 112 are configured to distribute the current over a wider area than the point source provided by the source electrode 106 and the point sink provided by the sink electrode 110.

Conventional neuromodulation devices like that of FIG. 1 are configured to apply a constant electrical current until the treatment is over. Additionally, no built-in safety mechanism exists to stop or adjust the current during treatment. Accordingly, conventional neuromodulation devices cause painful shocks when the current density rises in the gel pads during a pad peel event.

As discussed further below, physical arrangements and/or methods described with reference to embodiments herein may address the user discomfort caused by the current density shown in FIG. 1. Moreover, physical arrangements and/or methods described herein may improve user comfort as well as therapeutic effectiveness of a neuromodulation device without substantially increasing the physical dimension of the neuromodulation device, improving wearability and further increasing user comfort.

### Overview of Neuromodulation Device and Method of Operation

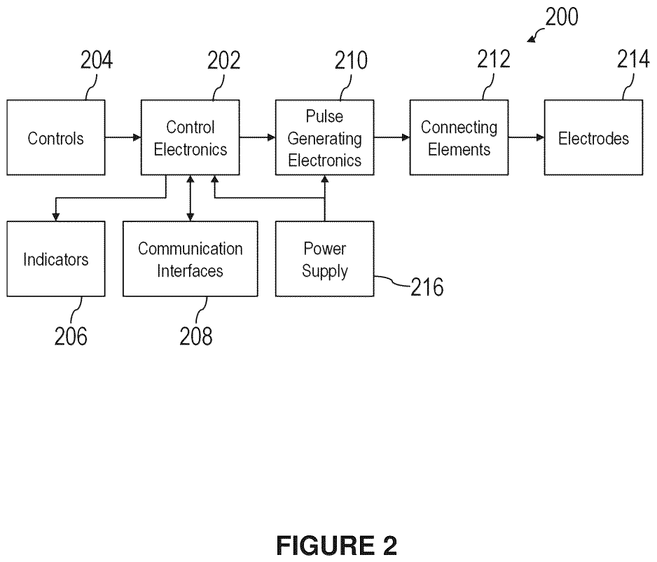

FIG. 2 is a block diagram illustrating modules of a neuromodulation device 200, according to some embodiments. The control electronics module 202 generally comprises a microcontroller which may be programmable to provide the desired pulse signals to the patient. The control electronics module 202 may also include the various electronics which are used to effect the treatment, e.g., timers, clocks, DACs, etc. which control the output on/off state, pulse amplitude, timing, modulation, and other pulse parameters. In order to actuate and/or interface with the control electronics module 202, a controls module 204 may be in communication with the control electronics module 202 through any number of interface mechanisms, e.g., buttons, knobs, sliders, capacitive touch sensors, etc. through which a user can turn the device on/off, adjust amplitude or other settings, etc. Additionally, the controls module 204 or other controller may communicate locally or remotely with the control electronics module 202 through a communication interface module 208 which may include any number of various wired and/or wireless communication mechanisms, e.g., Bluetooth®, Bluetooth® Low Energy or Bluetooth® Smart (Bluetooth SIG, Inc., Kirkland, Wash.), ANT, ZigBee, WiFi, infrared (e.g., Infrared Data Association (IrDA) associated wireless communications, etc.

The control electronics module 202 may provide any number of details, feedback, or information about its operation through an indicator module 206 which may include any variety of indicators, e.g., LEDs or indicator lights, displays such as LCDs, segment displays, etc., which may be positioned directly upon the neuromodulation device or separately in communication with the control electronics module 202.

In order for the neuromodulation device to provide the electrical stimulation to the patient's body, the control electronics module 202 may be in communication with a pulse generating electronics module 210 which is in communication with a electrodes 214 through a connecting elements 212. The control electronics module 202 and pulse generating electronics module 210 may be in communication with a power supply module 216 which supplies the power for the electrical stimulation. The power supply module 216 may include a battery such as a lithium-ion battery with associated circuits such as voltage regulators, LDOs, boost or buck converters, etc. The device output may utilize a voltage which is much higher than that available from the battery, and therefore the power supply may optionally include a generating mechanism for providing the high voltage as well as regulated low voltages for the other internal circuits.

The pulse generating electronics module 210 may include various components, including, but not limited to, amplifiers, op-amps, output filtering or pulse shaping circuits, output limiting or sensing and feedback circuits, elements which provide galvanic isolation, DC blocking, etc., which are configured to produce the electric pulses with controlled shape and amplitude as described herein.

Each of the various components may be in electrical communication through the connecting elements 212. The connecting elements used may comprise any number of electrically conductive elements, e.g., connectors, printed conductive traces, flex circuit boards, etc., which provide electrical connection from the electronics to the electrodes or between any number of electrical components.

The electrodes 214 electrically coupled to the pulse generating electronics module 210 may be shaped in various configurations for facilitating placement upon the patient depending upon the region of the body to be treated. Accordingly, the electrodes may be external for providing an electrical connection from the device output to the body through the skin, particularly to the target tissues or nerves.

External electrodes, in one variation, may be constructed of a conductive current-distributing element, an electrochemical electrode interface (such as a silver chloride coated silver, stainless steel, graphite, etc.) at which an electrochemical reaction may occur and a hydrogel (such as polyacrylamide or other stable and biocompatible gel with good adhesion) which contains a conductive solution (typically sodium chloride). The electrodes may be a driven as a pair of electrodes where the current flows from a first electrode to a second electrode, or as a more complex multi-polar setup, e.g., in a quadrupolar setup, with four electrodes driven as any one of six alternating pairs.

In other variations, the neuromodulation device may additionally and/or optionally include additional features or elements. For example, in one variation, the device may be controlled entirely via a communication interface using, e.g., wireless communication from a controller located remotely from the neuromodulation device. Such remotely located controllers may include, e.g., smartphones or other programmable devices, which may communicate via any number of wireless communication protocols, e.g., Bluetooth® Low Energy interface. Such a variation may remove the need for any controls or indicators on the device itself as the controls module 204 may be located remotely. In yet another alternative, the device may directly incorporate the controls module 204 upon the neuromodulation device itself so that it may be controlled entirely through an interface located upon the device.

Additionally, and/or alternatively, the control electronics module 202 and power supply module 216 may be packaged as a compact device which may be removably attached as a unit upon electrodes 214 which are disposable, e.g., polyacrylamide hydrogel with silver ink conductive traces printed on a polymer film base. The electrodes 214 may be placed upon the region of interest upon the patient body and the device may be temporarily coupled to an engagement mechanism which also allows for the electrical communication between the pulse generating electronics module 210 and the electrodes 214 to effect treatment upon the patient. This variation as well as others described may be combined in any number of combinations as practicable.

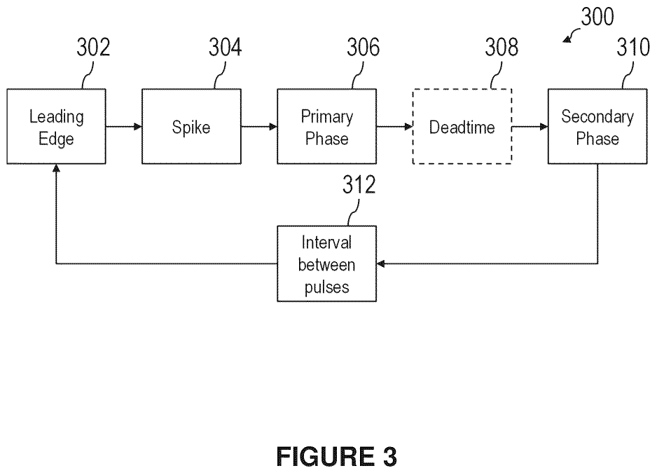

FIG. 3 is a block diagram illustrating phases of an electrical pulse output from a neuromodulation device, according to some embodiments. Specifically, FIG. 3 shows a block diagram of a representative pulse waveform 300 illustrating the major phases of an electrical pulse output. Using the neuromodulation device 200 of FIG. 2, the control electronics module 202 may be programmed to affect a specified pulse waveform generated by the pulse generating electronics module 210 and transmitted through the electrodes 214 and to the area of the patient's body upon which the electrodes 214 are positioned for treatment. Generally, the pulse waveform 300 may have a primary phase 306 followed by an optional dead time 308 period and then a secondary phase 310. Initiating the primary phase 306 is a leading edge 302 having a relatively fast rise time which leads to a spike 304 having an intensity greater than an average intensity of the primary phase 306. The remainder of the primary phase 306 may have an intensity which is lower than the intensity of the spike 304. In some embodiments, the remainder of the primary phase 306 may have an intensity approximately half intensity of the spike 304.

The dead time 308 period, if included, may have an output amplitude of zero. If the dead time 308 period is omitted, the secondary phase 310 may follow immediately after the primary phase 306 where the secondary phase 310 may have a polarity opposite to that of the primary phase 306. Like the dead time 308 period, the secondary phase 310 may be optionally omitted entirely from the pulse waveform 300. The treatment pulses having the pulse waveform 300 may be repeated during a treatment where a specified time interval period 312 may be present between each individual pulse waveform 300.

While the output is described here in terms of amplitude, this may include a measure of either current or voltage. In one embodiment, the pulse waveforms 300 may be produced by a circuit which is a voltage-limited current source, and the amplitudes may comprise current amplitudes. Using a current control allows for the effective movement of charges to be less dependent on the electrode impedance (which may change with skin condition or over time) and less dependent on the tissue impedance (which may change with placement or individually).

In another embodiment, the output may also comprise a voltage source or current-limited voltage source, since in the short term the tissue and electrode impedances are relatively constant and so the current is approximately equal to the voltage times a constant factor. In this case, the output may require more frequent adjustments. However, controlled or produced, the amplitude pattern shown describes the variation in electrical field strength independent of the effects of variation in electrode impedance or the specifics of the control circuit.

If any parameters (such as timings or amplitude) of the output electrical pulses depend on the load impedance, they may be measured using a resistive-capacitive test load simulating the electrodes and human body.

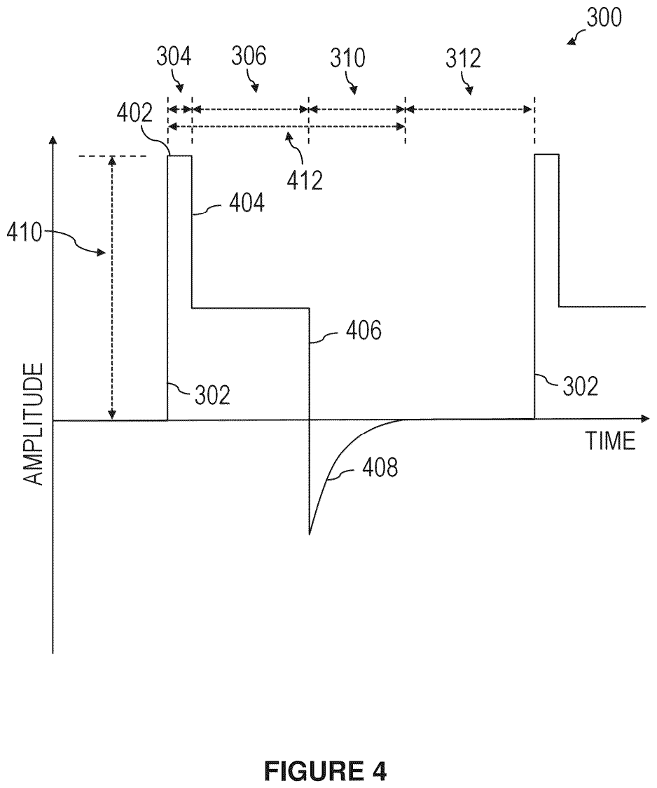

FIG. 4 graphically illustrates an individual representative pulse waveform 300 described in FIG. 3 with each of the major phases of an electrical pulse output. The primary phase 306 is shown including the leading edge 302 of spike 304 over a relatively short rise time and the remainder of the primary phase 306 where the intensity of the spike 304 is significantly greater than an average intensity of the primary phase 306 and where the intensity of the remainder is significantly lower than the intensity of the spike 304, e.g., about half the intensity. This may then be followed by the secondary phase 310 which may have its polarity opposite to that of the primary phase 306, as shown.

The transition or edge rise and fall times are measured as the time from, e.g., 10% to 90% of the change from initial to final level. Widths are measured as the time from, e.g., the 10% level on the rising edge to the 10% level on the falling edge. The illustration of the pulse waveform 300 is intended to be illustrative of the relative shape of the waveform with its timing measurements. Hence, the amplitudes during the spike 304, primary phase 306 and secondary phase 310 of the pulse waveform 300 do not need to be held constant as shown in FIG. 4. Any pulse shape such that the spike 304 has a relatively fast rise time and high peak amplitude 410, and the typical amplitude during the remainder of the primary phase 306 after the spike 304 is significantly less than the spike 304, may provide an effective pulse waveform 300. For example, if it is easier for the pulse generating or control electronics to produce, the primary phase 306 and secondary phase 310 may be comprised of, e.g., piecewise segments of exponentially decaying waveforms (e.g., due to capacitor discharge or inductor decay), piecewise linear, trapezoidal, or any other shape. The spike 304 itself may be shaped as, e.g., a half-sine wave, sine, parabolic, or similar waveform, as it is not necessary for it to have a constant level (e.g., “flat top”).

Additionally, the transitions between the spike 304, primary phase 306, optional dead time, secondary phase 310, and interval period 312 do not need to have rapid rise and fall times, or in any case the rapid rise and fall times are not necessary for effectiveness. However, it may be desirable to have a relatively rapid and/or fall time 404 at the end of the spike 304 to increase patient comfort. Once the brief time at maximum amplitude 402 during the spike 304 has passed, it is unlikely that any high but sub-maximum amplitude would have a physiological effect; however, it may cause greater charging of the skin capacitance and thus skin discomfort and for that reason it is desirable for the spike 304 to transition to the lower level during the remainder of the primary phase 306 relatively quickly. The fall time 406 at the end of the primary phase 306 and the fall time 408 from secondary phase 310 to the interval period 312 state or inter-pulse interval may be implemented in different ways while still having a therapeutically effective pulse shape as described herein. For example, as shown in FIG. 4, the fall time 406 may be stepwise, and the fall time 408 may be exponential decay. Other arrangements may be implemented, in other embodiments. The pulse may have an overall time period 412.

Turning now to the individual portions of the pulse waveform 300, the spike 304 is comprised of a relatively high amplitude 410, short duration spike which has a leading edge 302 with a fast rise time. The effective electric field during the spike 304 is expected (for several reasons described in more detail below) to be high enough to be able to drive conformational changes of the cellular components which are targeted therapeutically directly, by the electrostatic forces acting on fixed charges or dipoles therein. A relatively high electric field strength is desirable in order to produce conformational changes and thereby modulate the physiological function of the target cellular components or molecules; hence, an initial spike 304 with an amplitude 402 which is significantly higher than the average amplitude during the primary phase 306.

The transition between spike 304 and primary phase 306 may not be as fast as the leading edge 302 of the spike 304. In one embodiment, the transition is a rapid linear transition with a controlled fall time equal to the rise time of the leading edge 302 of the spike 304, but this is not required. Many variations in the transition between the spike 304 and primary phase 306 are possible, which accomplish the same goal of relatively rapid transition using different profiles.

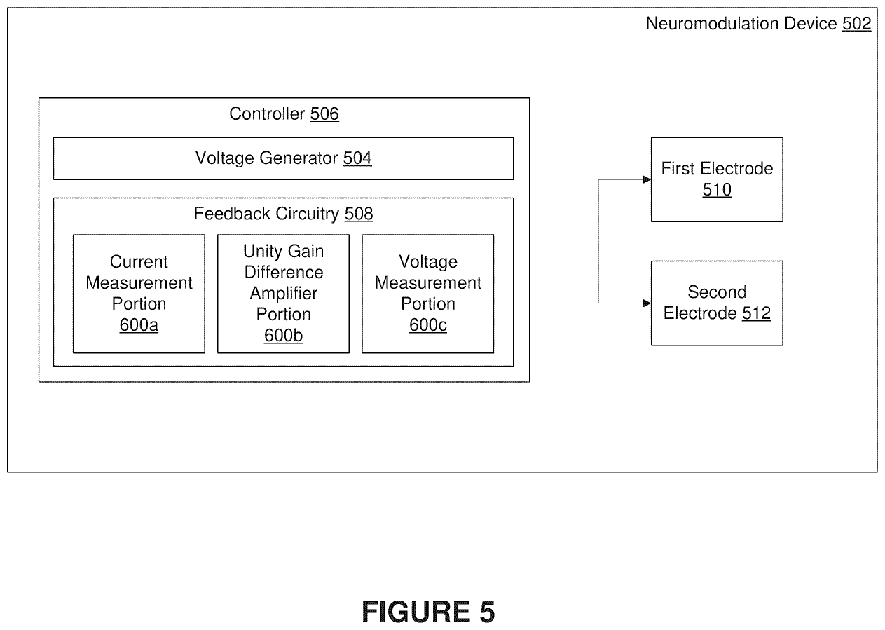

FIG. 5 is a block diagram illustrating an overview of the circuitry of a neuromodulation device 502 according to some embodiments. The neuromodulation device 502 includes a voltage generator 504, controller 506, and electrodes 510, 512. The voltage generator 504 is supplied power from a power source on the neuromodulation device 502, such as a battery. The voltage generator 504 may supply a voltage that the controller 506 uses to supply a current to the first electrode 510.

Controller 506 can use analog circuitry that controls the amount of current supplied to the first electrode 510. Controlling the amount of current enables the neuromodulation device to provide consistent treatment to a user. The controller 506 supplies the current as pulse waveforms to the first electrode 510. The controller 506 includes feedback circuitry 508. The feedback circuitry 508 enables the neuromodulation device 502 to measure a resistance value between the first electrode 510 and the second electrode 512. The feedback circuitry 508 measures the current supplied to the first electrode 510. The feedback circuitry 508 also measures the voltage between the first electrode 510 and the second electrode 512. Both the measured current and measured voltage difference values are captured with sample and hold circuits so that the current and voltage are measured within the same time window.

The feedback circuitry 508 sends the measured current and the measured voltage to a processor, where the resistance is calculated by dividing the voltage by the current (Ohm's law). A resistance value is calculated in real-time (e.g. every 1, 5, 10 seconds). To determine an average resistance, the processor can average the resistance value over a predetermined period, such as 5, 10, 30, and/or 60 seconds). The controller 506 can determine the existence of a pad peel event (e.g., the removal of a pad to which either or both the first electrode 510 and the second electrode 512 are attached) when there is a threshold delta or difference between the real-time resistance value and average resistance. This detection occurs continuously in real time once treatment by the neuromodulation device has started. The delta between the resistance value and the average resistance is used because the resistance value is constantly changing and/or drifting, so it is difficult to determine a higher-than-normal resistance value without comparing the real-time resistance level to the average resistance level.

A pad peel event, for example, occurs when there is a threshold difference between the resistance value and the average resistance. The difference in resistance can be caused when the current density rises due to the removal of the pad that the first electrode 510 and/or the second electrode 512 are attached to. When a pad peel event is detected, the neuromodulation device adjusts the current levels to prevent the user from experiencing a painful and uncomfortable shock. For example, when a threshold resistance delta is reached, the controller 506 can lower the intensity of the pulse waveforms used for treatment to prevent a shock from occurring due to a pad peel event. The intensity of the pulse waveforms can be decreased in proportion to the increase in the resistance, which is based on the degree or amount the pad is removed from the skin of the user. Additionally, controller 506 can stop treatment by halting the generation of the pulse waveforms when the resistance delta reaches the threshold value or when the resistance delta reaches a second greater resistance delta.

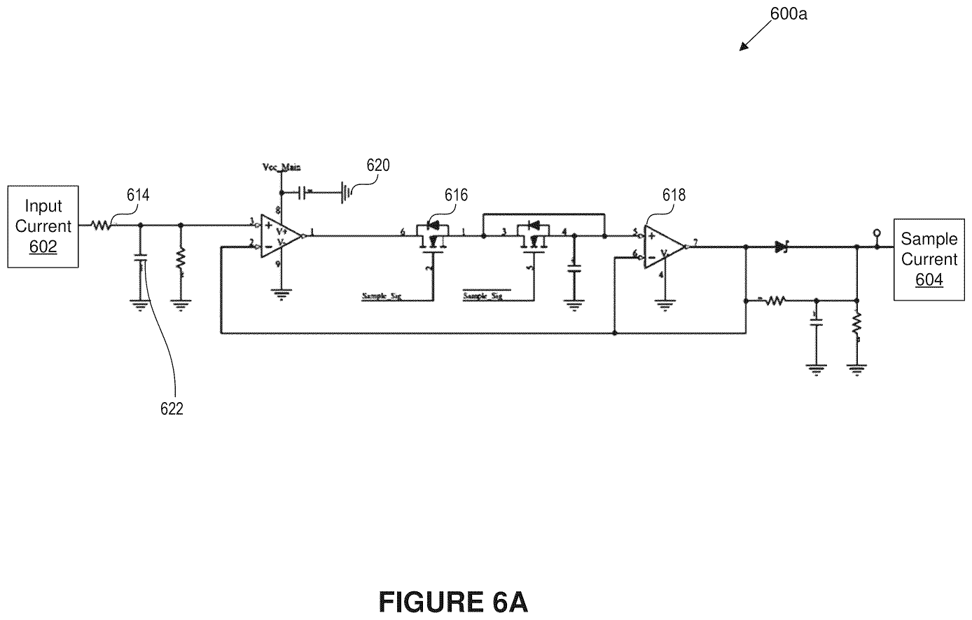

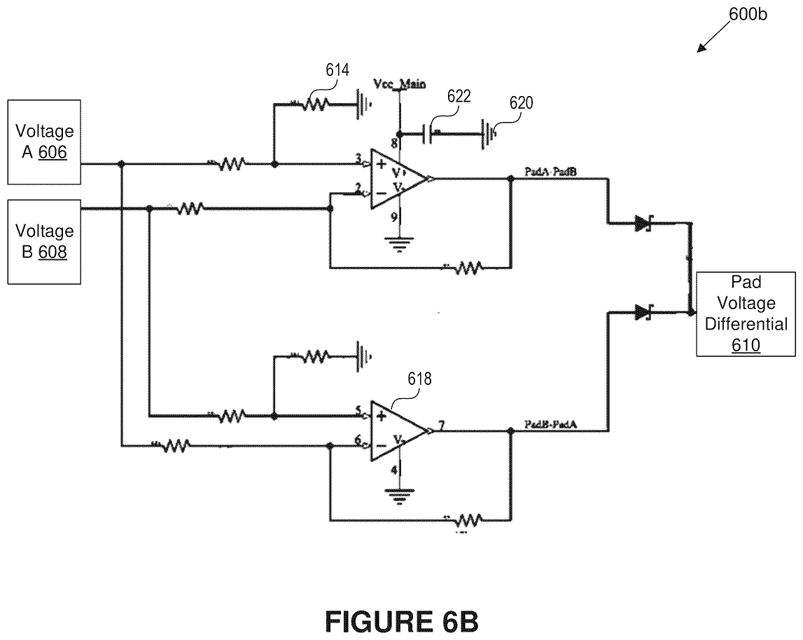

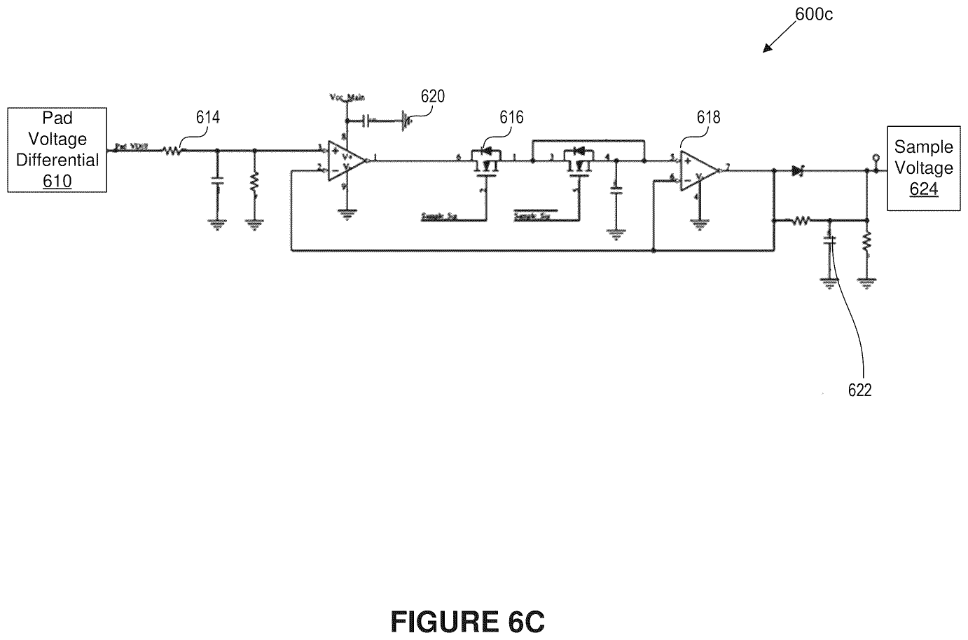

FIG. 6A is a circuit diagram illustrating the current measurement portion 600a of the feedback circuitry 508 of the neuromodulation device, according to some embodiments. The current measurement portion 600a of the feedback circuitry 508 enables the measurement of the current value used to calculate the resistance values used in determining the existence of a pad peel event. The current measurement portion 600a of the feedback circuitry 508 used to measure the sample current 604 can include multiple resistors 614, capacitors 622, grounds 620, operational amplifiers 618, and N-channel metal-oxide semiconductor field-effect transistor (MOSFET) 616. An operational amplifier can be used as a voltage amplifying device designed to be used with external feedback components such as resistors and capacitors between its output and input terminals. A MOSFET can change conductivity with the amount of applied voltage which can be used for amplifying or switching electronic signals. The current measurement portion can include a negative feedback loop to help maintain a consistent current pulse waveform. A voltage representing the real-time treatment current 602 flows through the current measurement portion 600a and is outputted as a sample voltage representing the magnitude of the current, here called the sample current 604. The controller uses the sample current 604 to calculate the resistance level between the first and second electrodes.

FIG. 6B is a circuit diagram illustrating a unity gain difference amplifier portion 600b of the feedback circuitry 508, according to some embodiments. The unity gain difference amplifier portion 600b is used to calculate the difference between voltage A 606 and a voltage B 608. Voltage A 606 is the voltage measured at the first electrode. Voltage B 608 is the voltage measured at the second electrode. The unity gain difference amplifier portion 600b includes multiple resistors 614, capacitors 622, grounds 620, and operational amplifiers 618. A pad voltage differential 610 is outputted by the unity gain difference amplifier portion 600b to be used by the voltage measurement portion 600c.

FIG. 6C is a circuit diagram illustrating a voltage measurement portion 600c of the feedback circuitry 508, according to some embodiments. The voltage measurement portion 600c of the feedback circuitry 508 enables the measurement of the voltage value used to calculate the resistance values used in determining the existence of a pad peel event. The voltage measurement portion 600c of the feedback circuitry 508 used to measure the sample voltage 624 can include multiple resistors 614, capacitors 622, grounds 620, operational amplifiers 618, and N-channel MOSFET 616. The voltage measurement portion 600c can be a sample and hold circuit to stabilize the voltage and increase the accuracy and consistency of the voltage measurement. The pad voltage differential 610 is inputted into the voltage measurement portion 600c, and the sample voltage 624 is outputted. The controller uses the sample voltage 624 to calculate the resistance level between the first and second electrodes.

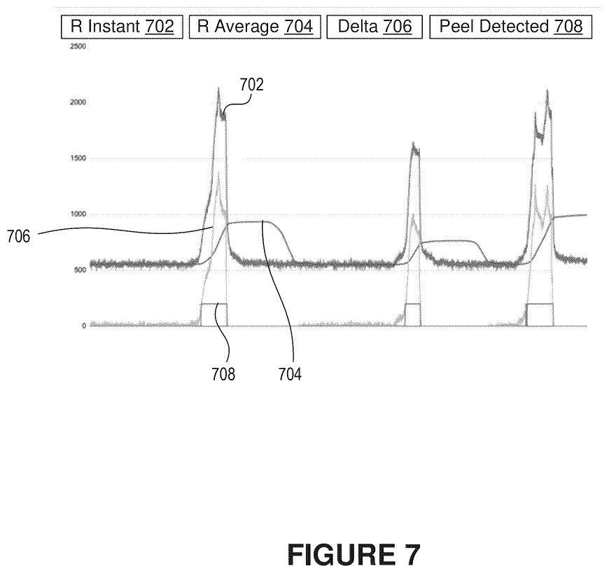

FIG. 7 is a graph illustrating sample resistance measurements according to some embodiments. The graph illustrates multiple pad peal events detected through the real-time continuous measurement of the resistance of the user's body. Resistance value 702 is the measured instant resistance as measured on the feedback circuitry 508. Resistance value 702 fluctuates and varies over time. The average resistance 704 is the resistance value 702 averaged over the predetermined time period. The average resistance 704 follows the resistance value 702. Delta 706 is the difference between the resistance value 702 and the average resistance 704. Due to the fluctuations of the resistance value 702, the delta 706 remains near zero unless a pad peel event occurs. Detection line 708 indicates that the delta 706 reached the threshold value to register as a detected pad peel event. Because the delta 706 is used to detect pad peel events, pad peel events can be detected in real-time at various resistance values and amplitudes.

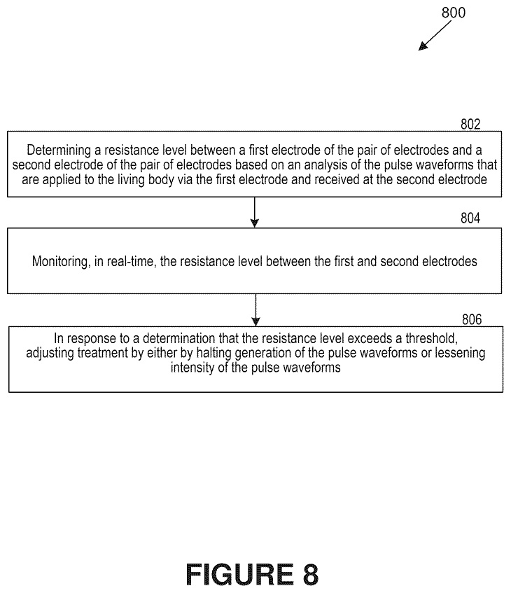

FIG. 8 is a flow diagram for a method 800 of detecting the removal of a neuromodulation device from the skin of a living body, according to some embodiments. The neuromodulation device includes a pair of electrodes through which pulse waveforms are applied to the living body. The electrodes are placed on an anatomical region of the living body. At 802, the method includes determining a resistance level between a first electrode of the pair of electrodes and a second electrode of the pair of electrodes based on an analysis of the pulse waveforms that are applied to the living body via the first electrode and received at the second electrode. When determining the resistance level between the first electrode and the second electrode, the method may further include measuring the electrical signal at the second electrode, where the electrical signal is a current value, and the current value is measured from an analog output using a feedback hardware. The method may further include calculating the resistance level based on the measured current value. At 804, the method includes monitoring, in real-time, the resistance level between the first and second electrodes. The method may further include averaging the resistance level between the pair of electrodes over a predetermined time period. At 806, the method includes, in response to a determination that the resistance delta level exceeds a threshold, adjusting treatment by either by halting generation of the pulse waveforms or lessening intensity of the pulse waveforms. The amount by which the intensity is adjusted may be proportional to the amount of surface area of the pad that has peeled so as to maintain the same current density and, therefore, not cause any change in the sensation. The threshold value may correspond to an increase in the resistance level within a threshold time period.

### REMARKS

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling those skilled in the relevant art to understand the claimed subject matter, the various embodiments, and the various modifications that are suited to the particular uses contemplated.

Although the Detailed Description describes certain embodiments and the best mode contemplated, the technology can be practiced in many ways no matter how detailed the Detailed Description appears. Embodiments can vary considerably in their implementation details, while still being encompassed by the specification. Particular terminology used when describing certain features or aspects of various embodiments should not be taken to imply that the terminology is being redefined herein to be restricted to any specific characteristics, features, or aspects of the technology with which that terminology is associated. In general, the terms used in the following claims should not be construed to limit the technology to the specific embodiments disclosed in the specification, unless those terms are explicitly defined herein. Accordingly, the actual scope of the technology encompasses not only the disclosed embodiments, but also all equivalent ways of practicing or implementing the embodiments.

The language used in the specification has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various embodiments is intended to be illustrative, but not limiting, of the scope of the technology as set forth in the following claims.

1. A neuromodulation device comprising:
a first electrode via which an electrical signal is applied to the skin of a living body at a first location;
a second electrode at which the electrical signal is received after traveling through an anatomical region of the living body;
a gel pad that is electrically connected with the first and second electrodes and is configured to be placed in contact with the skin of the living body; and
a controller configured to:
generate the electrical signal that is applied to the skin of the living body at the first location via the first electrode,
measure, using analog circuitry, the electrical signal received at the second electrode and produce an analog output that is representative of the measured electrical signal,
establish a change in resistance between the first and second electrodes based on the analog output,
determine whether the gel pad is being removed from the skin of the living body based on the change in resistance, and
halt generation of the electrical signal in response to the determination that the gel pad is being removed from the skin of the living body.

2. The neuromodulation device of claim 1, wherein prior to halting the generation of the electrical signal, the controller is further configured to:
decrease, until a threshold change resistance is met, an intensity of the electrical signal based on a degree the gel pad is removed from the skin of the living body.

3. The neuromodulation device of claim 1, wherein the controller includes:
a processor, and
an analog-to-digital converter configured to convert the analog output measured by the analog circuitry to a digital output that is receivable by the processor.

4. The neuromodulation device of claim 1, wherein to establish the change in resistance between the first and second electrodes, the controller is further configured to:
calculate a resistance value based on a measured current of the analog output.

5. The neuromodulation device of claim 4, wherein to establish the change in resistance between the first and second electrodes, the controller is further configured to:
determine, using the analog output, an average resistance between the first electrode and the second electrode, and
detect a spike in resistance between the first electrode and the second electrode.

6. The neuromodulation device of claim 5, wherein the resistance between the first electrode and the second electrode varies between users.

7. The neuromodulation device of claim 6, wherein a material of the gel pad affects the average resistance between the first electrode and the second electrode.

8. A method of detecting removal, from the skin of a living body, of a neuromodulation device having a pair of electrodes through which pulse waveforms are applied to the living body, the method comprising:
monitoring a resistance level between a first electrode of the pair of electrodes and a second electrode of the pair of electrodes based on an analysis of the pulse waveforms that are applied to the living body via the first electrode which are received at the second electrode;
calculating, at predetermined intervals, a resistance delta between the resistance level and an average resistance level between the pair of electrodes; and
in response to a determination that the resistance delta exceeds a threshold level,
adjusting treatment by either halting generation of the pulse waveforms or lessening an intensity of the pulse waveforms.

9. The method of claim 8, further comprising:
averaging the resistance level between the first and second electrodes over a predetermined time period.

10. The method of claim 9, further comprising:
comparing the resistance level to the average resistance level.

11. The method of claim 8, wherein the threshold level corresponds to an increase in the resistance delta over a threshold time period.

12. The method of claim 8, wherein monitoring the resistance level between the first electrode and the second electrode, further comprises:
determining a current value at the second electrode,
wherein the current value is measured from an analog output using feedback hardware;
measuring a voltage value between the pair of electrodes; and
calculating the resistance level based on the current value and the voltage value.

13. The method of claim 12, wherein further comprising:
amplifying a first input voltage and a second input voltage to increase a differential between the first input voltage and the second input voltage to enable measurement of the voltage value.

14. A device for treating a living body through electrical neuromodulation, the device comprising:
a first electrode via which an electrical signal is to be applied to the skin of a living body at a first location;
a second electrode at which the electrical signal is received after traveling through an anatomical region of the living body;
a gel pad that is electrically connected with the first and second electrodes and is configured to be placed in contact with the skin of the living body; and
a controller configured to:
generate the electrical signal that is applied to the skin of the living body at the first location via the first electrode,
measure, using analog circuitry, the electrical signal received at the second electrode and produce an analog output that is representative of the measured electrical signal,
establish a change in resistance between the first and second electrodes based on the analog output, and
in response to a determination, based on the change in resistance, that the gel pad is being removed from the skin of the living body,
halt generation of the electrical signal.

15. The device of claim 14, wherein prior to halting the generation of the electrical signal, the controller is further configured to:
decrease, until a threshold change in resistance is met, an intensity of the electrical signal based on a degree the gel pad is removed from the skin of the living body.

16. The device of claim 14, wherein the controller includes:
a processor, and
an analog-to-digital converter configured to convert the analog output measured by the analog circuitry to a digital output that is receivable by the processor.

17. The device of claim 14, wherein to establish the change in resistance between the first and second electrodes, the controller is further configured to:
calculate a resistance value based on a measured current of the analog output.

18. The device of claim 17, wherein to establish the change in resistance between the first and second electrodes, the controller is further configured to:
determine, using the analog output, an average resistance between the first electrode and the second electrode, and
detect a spike in resistance between the first electrode and the second electrode.

19. The device of claim 18, wherein the resistance between the first electrode and the second electrode varies between users.

20. The device of claim 19, wherein a material of the gel pad affects the average resistance between the first electrode and the second electrode.
